(19) United States
(12) Patent Application Publication
(10) Pub. No.: US 2019/0053997 A1
(43) Pub. Date: Feb. 21, 2019

(54) SKIN CARE COSMETIC SET AND APPLICATION METHOD THEREOF

(71) Applicant: Yumei LIU, New York, NY (US)

(72) Inventor: Yumei LIU, New York, NY (US)

(21) Appl. No.: 15/679,140

(22) Filed: Aug. 16, 2017

(51) Int. Cl. A61K 8/73; A61K 8/34; A61K 8/67; A61K 8/41; A61K 8/29; A61Q 19/10

(52) U.S. Cl. CPC A61K 8/735; A61K 8/342; A61K 8/678; A61K 2800/884; A61K 8/29; A61Q 19/10; A61K 8/416

(57) ABSTRACT

The present disclosure relates to a skin care cosmetic set. The skin care cosmetic set comprises a facial cleanser, a skin care toner, a serum, a whitening cream and a protective emulsion. The facial cleanser comprises an olive extract and a wheat extract; the skin care toner comprises sodium hyaluronate, a hamamelis virginiana extract and betaine; the serum comprises an aloe extract, a soybean extract, EGF moisturizing factors and sodium hyaluronate; the whitening cream comprises cetyl alcohol, vaseline, vitamin E and sodium hyaluronate; the protective emulsion comprises a wheat extract, glycerol, alpha-bisabolol and nano-titanium dioxide, and also discloses an application method thereof. The present invention is used by combinations to form a multi-layer skin care layer, and each skin care layer cooperate with each other, so that the skin care has more comprehensive effect.

### TECHNICAL FIELD

The present invention relates to skin care cosmetics, and more specifically, to a skin care cosmetic set and application method thereof.

### BACKGROUND OF THE INVENTION

With the continuous improvement of living standards, people's pursuit of beauty is more and more intense, skin care and skin beauty have become the great social needs, and more and more people are using skin care cosmetics, the ideal skin care cosmetics should be less pure impurities, good stability, no irritation to the skin, little allergic reaction or pigment change, no absorption of poison in long term use, and no harm to the skin. Proper use of skin care cosmetics can play the role of protecting the skin and delaying the aging of the skin, on the contrary, however, improper use can also cause harm to the skin, leading to premature aging or other skin diseases. Therefore, in order to meet the needs of the market, many businesses produce a variety of different brands of skin care cosmetics, but in many cases people can not distinguish between different types of skin care cosmetics, and even the situation that improper use result in dermatitis and pigmentation often happens.

However, there are many kinds of skin care cosmetics, which can be divided into cream, lotion, powder, liquid and gel according to their form; and can be divided into cleaning, moisturizing, beautifying, whitening, anti-aging, etc. according to the use purpose, a single effect of skin care cosmetics can not meet people's demand, different brands and types of skin care cosmetics increase the possibility of repeated choice about ingredients, and its mixed use increases the risk of improper use.

### SUMMARY OF THE INVENTION

In view of the deficiencies of the above-mentioned techniques, it is an object of the present invention to provide a skin care cosmetic set and application method thereof, in which the efficacy and use purpose of each individual product are different and coordinate with each other by working together. According to the method of the present invention, the skin can be cleaned, moisturized, improved, repaired, isolated and protected if used in regular manner. The present invention is achieved by the following technical scheme: a skin care cosmetic set, comprising: a facial cleanser, a skin care toner, a serum, a whitening cream and a protective emulsion. The facial cleanser comprises following ingredients by mass percent: an olive extract 1%-10%, and a wheat extract 1%-10%; the skin care toner comprises following ingredients by mass percent: sodium hyaluronate 5%-10%, a hamamelis virginiana extract 1%-15%, and betaine 1%-10%; the serum comprises following ingredients by mass percent: an aloe extract 8%-10%, a soybean extract 10%-20%, EGF moisturizing factors 0.001%-0.01%, and sodium hyaluronate 1%-6%; the whitening cream comprises following ingredients by mass percent: cetyl alcohol 2%-4%, vaseline 10%-20%, vitamin E 1%-5%, and sodium hyaluronate 7%-15%; and the protective emulsion comprises following ingredients by mass percent: a wheat extract 10%-15%, glycerol 1%-2%, alpha-bisabolol 0.2%-0.5%, and nano-titanium dioxide 22%-32%.

Preferably, the facial cleanser further comprises sodium laureth sulfate, alkyl sulfonated stearate, lauryl phosphate, sodium N-lauroyl sarcosinate, alkyl polyglucoside, sodium cocoyl isethionate, ethylene glycol distearate and methyl isobutyl thiazolinone.

Preferably, the skin care toner further comprises glycerol, butanediol, dextran, allantoin and chlorphenesin.

Preferably, the serum further comprises propylene glycol, 1,3-butanediol, nicotinamide, trimethylglycine, spirulina proteoglycans, cellulose, carbomer, triethanolamine, methyl hydroxybenzoate and 1,2-Benzisothiazolin-3-One.

Preferably, the whitening cream further comprises polydimethylsiloxane, octanoic acid, petrolatum, cyclodimethylsiloxane, sorbitan stearate, cetostearyl alcohol, stearic acid, nicotinamide, allantoin, diazolidinyl urea and arginine.

Preferably, the protective emulsion further comprises propylene glycol, sodium chloride, decamethylcyclopentasiloxane, polydimethylsiloxane squalane, cetyl polyethylene glycol, zinc oxide and iodopropynylbutyl carbamate.

Preferably, the wheat extract of the cleanser includes wheat germ oil and a water-soluble extract of wheat germ.

Preferably, the soybean extract of the serum includes hydrolyzed soybean protein, soybean lecithin and soybean isoflavones.

Preferably, the nano-titanium dioxide of the protective emulsion has a particle size of 40 nm to 80 nm.

An application method of a skin care cosmetic set, the skin care cosmetic set comprises: a facial cleanser, a skin care toner, a serum, a whitening cream and a protective emulsion; and the application method comprises the following steps:

(a) cleaning skin with the facial cleanser, and washing with water;

(b) wetting the skin with the skin care toner;

(c) further wetting the skin by using the serum;

(d) smearing the whitening cream on the wet skin; and

(e) further smearing the protective emulsion on the wet skin.

The facial cleanser comprises following ingredients by mass percent: an olive extract 1%-10%, and a wheat extract 1%-10%; the skin care toner comprises following ingredients by mass percent: sodium hyaluronate 5%-10%, a hamamelis virginiana extract 1%-15%, and betaine 1%-10%; the serum comprises following ingredients by mass percent: an aloe extract 8%-10%, a soybean extract 10%-20%, EGF moisturizing factors 0.001%-0.01%, and sodium hyaluronate 1%-6%; the whitening cream comprises following ingredients by mass percent: cetyl alcohol 2%-4%, vaseline 10%-20%, vitamin E 1%-5%, and sodium hyaluronate 7%-15%; the protective emulsion comprises following ingredients by mass percent: a wheat extract 10%-15%, glycerol 1%-2%, alpha-bisabolol 0.2%-0.5%, and nano-titanium dioxide 22%-32%.

The beneficial effect of the present invention is to use the set in combination, each of the set focuses on the different effects and functions. The facial cleanser can clean as well as deeply cure and repair facial skin at the same time. the skin care toner is focused on the moisturizing, healing and preventing scar effect on epidermal injury; the serum further strengthens the moisturizing layer and forms a underlying protective repair layer; the whitening cream protects the moisturizing layer, locks the water and form sa medium matrix layer; the protective emulsion is used to form the outer insulation layer, focusing on isolation and protection. Through the combination of using the set, a multi-layer skin layer is formed, and each skin care layer cooperate with each other, so that the skin care has more comprehensive effect.

### DETAILED DESCRIPTION OF THE INVENTION

The present invention will be described in more detail in the following examples, but the invention is not limited in any way.

### Example 1

The present embodiment provides a skin care cosmetic set, comprising: a facial cleanser, a skin care toner, a serum, a whitening cream and a protective emulsion, the facial cleanser comprises 2% (wt) of olive extracts and 8% (wt) of wheat extracts, and further comprises sodium laureth sulfate, alkyl sulfonated stearate, lauryl phosphate, sodium N-lauroyl sarcosinate, alkyl polyglucoside, sodium cocoyl isethionate, ethylene glycol distearate and ethylene glycol distearate. And the facial cleanser of the present embodiment is obtained by mixing above ingredients evenly into an emulsion with appropriate amount of deionized water.

The skin care toner comprises 6% (wt) of sodium hyaluronate, 5% (wt) of Hamamelis virginiana extract and 3% (wt) of betaine, and further comprises glycerol, butanediol, dextran, allantoin and chlorphenesin. And the skin care toner of the present embodiment is obtained by mixing above ingredients into a liquid with appropriate amount of deionized water.

The serum comprises 8% (wt) of aloe extracts, 13% (wt) of soybean extracts, 0.005% (wt) of EGF moisturizing factors and 2% (wt) of sodium hyaluronate, and further comprises propylene glycol, 1,3-butanediol, nicotinamide, trimethylglycine, spirulina proteoglycans, cellulose, carbomer, triethanolamine, methyl hydroxybenzoate and 1,2-Benzisothiazolin-3-One. And the serum of the present embodiment is obtained by mix above ingredients into a liquid with appropriate amount of deionized water.

The whitening cream comprises 2% (wt) of cetyl alcohol, 13% (wt) of vaseline, 2% (wt) vitamin E and 9% (wt) of sodium hyaluronate, and further comprises polydimethylsiloxane, octanoic acid, petrolatum, cyclodimethylsiloxane, sorbitan stearate, cetostearyl alcohol, stearic acid, nicotinamide, allantoin, diazolidinyl urea and arginine. And the whitening cream of the present embodiment is obtained by mixing above ingredients into an emulsion with appropriate amount of deionized water.

The protective emulsion comprises 12% (wt) of wheat extracts, 1% (wt) of glycerol, 0.3% (wt) of alpha-bisabolol, and 25% (wt) of nano-titanium dioxide, and further comprises propylene glycol, sodium chloride, decamethylcyclopentasiloxane, polydimethylsiloxane squalane, cetyl polyethylene glycol, zinc oxide and iodopropynylbutyl carbamate. And the protective emulsion of the present embodiment is obtained by mixing above ingredients into an emulsion with appropriate amount of deionized water.

In the present embodiment, the olive extracts retains natural nutrient composition without heating and chemical processing, which can easily penetrate into deep skin, promote fat-soluble vitamins and antioxidants, etc., deep into the skin, nourish the skin, increase the gloss elasticity and delay the existence of wrinkles. The wheat extracts preferably includes wheat germ oil and a water-soluble extract of the wheat germ, the wheat germ oil is rich in bioactive oil components, has high efficiency antioxidant function promotes skin metabolism, enhances skin moisturizing, and hays a role in preventing pigmentation, delaying skin aging and reducing fine lines and scars. The wheat germ soluble water-soluble extract is rich in amino acids and trace elements, which can supplement the natural moisturizing factor of the skin, and combined with wheat germ oil, water replenishing and reserving of deep cells can be realized.

The sodium hyaluronate can prevent the formation of chapped skin and wrinkles and accelerate wound healing, which has moisturizing effect. Hamamelis Virginiana has a strong effect on skin cut, abrasions and sunburn, which is an antioxidant and astringent and is effectively to treat acne. Betaine has a strong hygroscopic property, it can play a very good cleaning effect without harming the skin, which is very safe.

The aloe extracts contain ingredients to eliminate superoxide radicals, such as superoxide dismutase and catalase, which can make the skin delicate, flexible, soft, moisturize, anti-inflammatory, improve scars, and have a strong absorption of ultraviolet light to prevent pigmentation and keep the skin white. The soybean extracts can prevent damage to the human body by free radicals and improve immunity, the soybean extracts include hydrolyzed soybean protein, soybean lecithin and soybean isoflavones. Hydrolyzed soybean protein induces skin to produce hyaluronic acid, which has a highly effective moisturizing effect. Soybean lecithin has capacity to beautify the skin and improve cell membrane self-repair. Soybean isoflavones are a natural nutrient with a variety of important physiological activities. EGF moisturizing factors enhances the vitality of epidermal cells and delays the aging of epidermal cells, and keeps all the components of the skin in their best physiological state.

Vaseline has a certain water absorption, which can be increase through mixing with cetyl alcohol, and emulsion matrix is formed in fully stirring after absorbing water, which has a stabilization and thickening effect, and a middle protective layer is formed with vitamin E and sodium hyaluronate.

Nano-titanium dioxide has strong effect on shielding ultraviolet, and can absorb ultraviolet light, which has good dispersion and weather resistance, and when its particle size is less than 100 nm, it is transparent, paste is delicate and with natural skin feeling. The particle size range of the nano-titanium dioxide is preferably 40 nm to 80 nm, and the skin whiteness is natural in this range. Nano-titanium dioxide also has anti-bacterial, self-cleaning and anti-aging effect, and an outer isolation layer is formed with wheat extracts, glycerol and alpha-bisabolol, which plays a role in replenishing the underlying ingredients through slowly penetration.

### Example 2

The present embodiment is based on the embodiment 1, except that the weight percent of the ingredients is different.

In the facial cleanser, 8% (wt) of olive extracts and 3% (wt) of wheat extracts are added; in the skin care toner, 8% (wt) of sodium hyaluronate, 12% (wt) of hamamelis virginiana extract and 8% (wt) of betaine are added; in the serum, 9% (wt) of aloe extracts, 19% (wt) of soybean extracts, 0.01% (wt) of EGF moisturizing factors and 5% (wt) sodium hyaluronate are added; in the whitening cream, 4% (wt) of cetyl alcohol, 18% (wt) of vaseline, 5% (wt) of vitamin E, and 14% (wt) of sodium hyaluronate are added; in the protective emulsion, 15% (wt) of wheat extracts, 2% (wt) of glycerol, 0.5% (wt) of alpha-bisabolol, and 30% (wt) of nano-titanium dioxide are added.

The application method of a skin care cosmetic set, comprising the following steps: (a) cleaning skin with the facial cleanser, and washing with water; (b) wetting the skin with the skin care toner; (c) further wetting the skin by using the serum; (d) smearing the whitening cream on the wet skin; and (e) further smearing the protective emulsion on the wet skin. The above steps are preferably repeated at least once a day.

The present invention integrates the composition of each product and the layered characteristics to improve the skin care effect.

It should be understood that any improvements and modifications made by one of ordinary skill in the art based on foregoing description are intended to be within the scope of the appended claims.

Above disclosure is merely illustrative description, it is obvious that the present invention is not limited thereto. Any improvements made based on the inventive concept and technical scheme of the present invention, or application directly on other occasions without improvements shall fall into the protection scope of the present invention.

What is claimed is:

1. A skin care cosmetic set, wherein the cosmetic set comprises: a facial cleanser, a skin care toner, a serum, a whitening cream and a protective emulsion;
the facial cleanser comprises following ingredients by mass percent: an olive extract 1%-10%, and a wheat extract 1%-10%;
the skin care toner comprises following ingredients by mass percent: sodium hyaluronate 5%-10%, a hamamelis virginiana extract 1%-15%, and betaine 1%-10%;
the serum comprises following ingredients by mass percent: an aloe extract 8%-10%, a soybean extract 10%-20%, EGF moisturizing factors 0.001%-0.01%, and sodium hyaluronate 1%-6%;
the whitening cream comprises following ingredients by mass percent: cetyl alcohol 2%-4%, vaseline 10%-20%, vitamin E 1%-5%, and sodium hyaluronate 7%-15%; and
the protective emulsion comprises following ingredients by mass percent: a wheat extract 10%-15%, glycerol 1%-2%, alpha-bisabolol 0.2%-0.5%, and nano-titanium dioxide 22%-32%.

2. The skin care cosmetic set of claim 1, wherein the facial cleanser further comprises sodium laureth sulfate, alkyl sulfonated stearate, lauryl phosphate, sodium N-lauroyl sarcosinate, alkyl polyglucoside, sodium cocoyl isethionate, ethylene glycol distearate and methyl isobutyl thiazolinone.

3. The skin care cosmetic set of claim 1, wherein the skin care toner further comprises glycerol, butanediol, dextran, allantoin and chlorphenesin.

4. The skin care cosmetic set of claim 1, wherein the serum further comprises propylene glycol, 1,3-butanediol, nicotinamide, trimethylglycine, spirulina proteoglycans, cellulose, carbomer, triethanolamine, methyl hydroxybenzoate and 1,2-Benzisothiazolin-3-One.

5. The skin care cosmetic set of claim 1, wherein the whitening cream further comprises polydimethylsiloxane, octanoic acid, petrolatum, cyclodimethylsiloxane, sorbitan stearate, Cetostearyl alcohol, stearic acid, nicotinamide, allantoin, diazolidinyl urea and arginine.

6. The skin care cosmetic set of claim 1, wherein the protective emulsion further comprises propylene glycol, sodium chloride, decamethylcyclopentasiloxane, polydimethylsiloxane squalane, cetyl polyethylene glycol, zinc oxide, iodopropynylbutyl carbamate.

7. The skin care cosmetic set of claim 1, wherein the wheat extract of the cleanser includes wheat germ oil and a water-soluble extract of wheat germ.

8. The skin care cosmetic set of claim 1, wherein the soybean extract of the serum includes hydrolyzed soybean protein, soybean lecithin and soybean isoflavones.

9. The skin care cosmetic set of claim 1, wherein the nano-titanium dioxide of the protective emulsion has a particle size of 40 nm to 80 nm.

10. An application method of a skin care cosmetic set, characterized in that the skin care cosmetic set comprises a facial cleanser, a skin care toner, a serum, a whitening cream and a protective emulsion, and the application method comprises the following steps:
(a) cleaning skin with the facial cleanser, and washing with water;
(b) wetting the skin with the skin care toner;
(c) further wetting the skin by using the serum;
(d) smearing the whitening cream on the wet skin; and
(e) further smearing the protective emulsion on the wet skin;
wherein the facial cleanser comprises following ingredients by mass percent: an olive extract 1%-10%, and a wheat extract 1%-10%;
the skin care toner comprises following ingredients by mass percent: sodium hyaluronate 5%-10%, a hamamelis virginiana extract 1%-15%, and betaine 1%-10%;
the serum comprises following ingredients by mass percent: an aloe extract 8%-10%, a soybean extract 10%-20%, EGF moisturizing factors 0.001%-0.01%, and sodium hyaluronate 1%-6%;
the whitening cream comprises following ingredients by mass percent: cetyl alcohol 2%-4%, vaseline 10%-20%, vitamin E 1%-5%, and sodium hyaluronate 7%-15%;
the protective emulsion comprises following ingredients by mass percent: a wheat extract 10%-15%, glycerol 1%-2%, alpha-bisabolol 0.2%-0.5%, and nano-titanium dioxide 22%-32%.
